(19) United States
(12) Patent Application Publication
(10) Pub. No.: US 2025/0205082 A1
(43) Pub. Date: Jun. 26, 2025

(54) Control of Irrigation in a Phacoemulsification System

(71) Applicant: JOHNSON & JOHNSON SURGICAL VISION, INC., Irvine, CA (US)

(72) Inventor: Vadim Gliner, Haifa (IL)

(21) Appl. No.: 18/390,785

(22) Filed: Dec. 20, 2023

(51) Int. Cl. A61F 9/007; A61M 3/02

(52) U.S. Cl. CPC A61F 9/00745; A61M 3/0202; A61M 3/022; A61M 3/0258; A61B 2217/005; A61B 2217/007; A61M 2205/0294

(57) ABSTRACT

A phacoemulsification system includes a handpiece, an irrigation module, an aspiration module, circuitry, and a processor. The handpiece includes a piezoelectric element and a needle. The needle is configured to be inserted into an eye and to be vibrated by the piezoelectric element to emulsify a lens of the eye. The irrigation module is configured to supply a flow of irrigation fluid to the eye based on respective values of at least one control parameter. The aspiration module is configured for aspiration of material from the eye. The circuitry is configured to determine a difference between the flow of irrigation fluid to the eye and an aspiration flow of the material from the eye. The processor is configured to set respective values of the at least one control parameter based on the determined difference and to provide the control parameter value(s) to the irrigation module.

### TECHNOLOGICAL FIELD

The present application relates to controlled operation of a phacoemulsification system, and more specifically to automated control of irrigation in a phacoemulsification system.

### BACKGROUND

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then, a portion of the anterior surface of the lens capsule is removed to gain access to the cataract. The surgeon then uses a phacoemulsification probe, which has an ultrasonic handpiece with a needle. The tip of the needle vibrates at ultrasonic frequency to sculpt and emulsify the cataract while a pump aspirates particles and fluid from the eye through the tip. After removing the cataract with phacoemulsification, the softer outer lens cortex is removed with suction. An intraocular lens is then introduced into the empty lens capsule restoring the patient's vision.

During the phacoemulsification procedure, the eye is irrigated with irrigation fluid (e.g. a balanced salt solution) to maintain intraocular pressure (IOP) within safe limits. Fluid lost from the eye includes fluid removed from the eye by aspiration (denoted herein aspiration flow) and fluid leaking from the eye through the incision or incisions made at the surgical site.

The rate of irrigation fluid flow should correspond closely to the loss of fluid from the eye in order to prevent either under-pressure or over-pressure in the eye, either of which can cause trauma. However, while the flow of irrigation fluid and the aspiration flow may be determined by the phacoemulsification system, the fluid loss through the incision(s) cannot be predicted and may vary over the duration of the procedure. Setting the irrigation flow to compensate for maximum leakage may create a high IOP which is not necessary for procedures with lower levels of leakage.

### BRIEF DESCRIPTION OF THE DRAWINGS

In order to better understand the subject matter that is disclosed herein and to exemplify how it may be carried out in practice, examples will now be described, by way of non-limiting example only, with reference to the accompanying drawings, in which:

FIG. 1 is a schematic, pictorial view of an exemplary phacoemulsification system, according to examples of the present disclosure;

FIG. 2 is a simplified block diagram of a phacoemulsification system, according to examples of the present disclosure;

FIG. 3 is a simplified flowchart of a method for controlling irrigation in a phacoemulsification system, according to examples of the present disclosure; and

FIG. 4 is a simplified diagram illustrating the gradual adjustment of parameter values for a PID controller over time.

### DETAILED DESCRIPTION OF EXAMPLES

In some examples, a phacoemulsification system is configured to operate in an adaptive mode, in which the rate of flow of irrigation fluid is adapted over time to compensate for fluid leakage from the eye through the incision in the eye (also known as wound leakage). In such systems, irrigation may be provided, decreased or increased by a controller based on parameter values that are based on the difference between flow of irrigation fluid and the aspiration flow.

According to some examples, the phacoemulsification system compensates for wound leakage using a feedback loop based on the difference between the flow of irrigation fluid and the aspiration flow from the eye. This difference is used to determine the values of one or more parameters that control the rate at which irrigation fluid is supplied to the eye.

According to some examples, the parameter values are determined from an average of the difference between irrigation fluid flow and aspiration flow over a preceding time period. Optionally, the duration of the time period is relatively long (for example one minute).

Adjusting the control parameter values as described herein achieves responsive control of the flow of irrigation fluid, while not risking the safety of the eye by large and/or rapid changes in the irrigation fluid flow rate. The inventors have found that under conditions of very low leakage, examples of the invention may provide for a 27 mm Hg IOP, as compared to the 70 mm Hg which is common when irrigation fluid flow is set to compensate for maximal leakage.

In the following detailed description, numerous specific details are set forth in order to provide a thorough understanding of the invention. However, it will be understood by those skilled in the art that the presently disclosed subject matter may be practiced without these specific details. In other instances, well-known methods, procedures, components and circuits have not been described in detail so as not to obscure the presently disclosed subject matter.

Unless specifically stated otherwise, as apparent from the following discussions, it is appreciated that throughout the specification discussions utilizing terms such as “processing”, “computing”, “comparing”, or the like, refer to the action(s) and/or process(es) of a computer that manipulate and/or transform data into other data, said data represented as physical, such as electronic, quantities and/or said data representing the physical objects. The term “computer” should be expansively construed to cover any kind of hardware-based electronic device with data processing capabilities including, by way of non-limiting example, a Fabrication Process Examination Information (FPEI) system and respective parts thereof disclosed in the present application.

### I. Phacoemulsification System

Generally, a phacoemulsification system includes a probe or a handpiece which comprises a needle and a sleeve that is at least partially insertable into an eye; a transducer such as a piezoelectric element that is configured to vibrate the needle for breaking the cataract into smaller particles; an aspiration module which aspirates the particles from the eye; an irrigation module which introduces fluid into the eye (for one or more of: (i) compensating for eye fluid aspirated from eye for maintaining intraocular pressure (IOP) in eye, (ii) controlling a temperature in the eye (e.g., by dissipating heat generated by the vibration of needle during the procedure), and (iii) facilitating the removal of lens particles from the eye); control means (e.g. a processor); and circuitry (for example sensing circuitry which comprises one or more sensors).

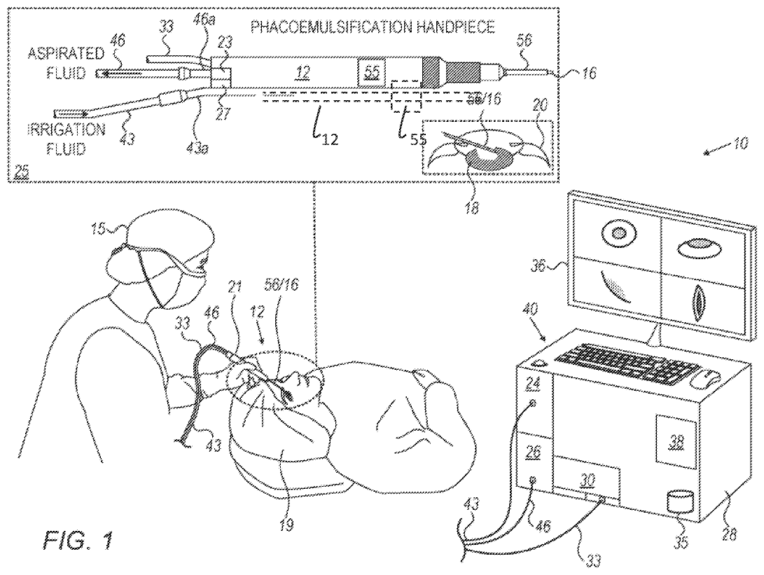

Reference is now made to FIG. 1, which is a schematic, pictorial view of an exemplary phacoemulsification system in which the flow of irrigation fluid may be controlled according to examples of the present disclosure.

As seen in the pictorial view of phacoemulsification system 10, and #n inset 25, phacoemulsification probe 12 (also referred to herein as a handpiece 12) comprises a needle 16 substantially surrounded by an irrigation sleeve 56. Needle 16 is hollow and its lumen is used as an aspiration channel.

Needle 16 is configured for insertion by a physician 15 into an eye 20 of a patient 19, for example into a lens capsule 18 of the eye, to remove a cataract. The needle 16 (and irrigation sleeve 56) are shown in inset 25 as a straight object. However, any suitable needle may be used with phacoemulsification probe 12, for example, a curved or bent tip needle commercially available from Johnson & Johnson Surgical Vision, Inc., Irvine, CA., USA.

Referring now to the irrigation and aspiration modules, irrigation channels 43a and aspiration channel 46a are coupled with irrigation tube 43 and aspiration tube 46, respectively.

Referring now to the system circuitry, and more specifically to sensing circuitry, in the shown example, probe 12 includes a sensor 27 coupled with irrigation channel 43a, and a sensor 23 coupled with aspiration channel 46a. It is noted that other examples may include only one sensor in either of the channels. Generally, one or more sensors may be located at any suitable position along the handpiece, and/or operably attached to the handpiece, such as at a module coupled with or attached at a proximal end of the handpiece.

Sensors 23 and 27 may be any sensor known in the art, including, but not limited to a vacuum sensor or flow sensor. The sensor measurements (e.g., pressure, vacuum, and/or flow) may be taken close to the distal end of the handpiece where the irrigation outlet and the aspiration inlet are located, so as to provide processor 38 an accurate indication of the actual measurements occurring within an eye and provide a short response time to a control loop comprised in processor 38. It is noted that other types of sensors such as temperature sensors, optical sensors, capacitance sensors and/or other sensors may also be incorporated as part of the circuitry of the system.

In some examples, system 10 comprises a processor-controlled irrigation pump 24. During the phacoemulsification procedure, processor-controlled pump 24 comprised in a console 28 pumps irrigation fluid from an irrigation reservoir or tank (not shown) to irrigate the eye via irrigation sleeve 56. The fluid is pumped via irrigation tubing line 43 running from console 28 to probe 12. Irrigation pump 24 may be any pump known in the art, for example, a peristaltic pump or a progressive cavity pump. In some examples, a gravity fed irrigation source such as a balanced salt solution bottle or bag may be used with pump 24 or in replacement of the pump.

In some examples, processor 38 controls a pump rate of irrigation pump 24, for example to maintain intraocular pressure within prespecified limits, to enable or disable irrigation, to increase or decrease a flow volume of irrigation fluid, to open or close valves along the irrigation line, or otherwise control irrigation.

In some examples, eye fluid and waste matter (e.g., emulsified parts of the cataract) are aspirated via hollow needle 16 to a collection receptacle (not shown) by a processor-controlled aspiration pump 26 also comprised in console 28 and using aspiration tubing line 46 running from probe 12 to console 28. In some examples, the same processor that operates the irrigation pump also operates the one or more aspiration pumps, and in some cases, simultaneously. In an example, processor 38 controls an aspiration rate of aspiration pump 26 to maintain intraocular pressure (in case of sub-pressure indicated, for example, by sensor 23) within prespecified limits.

As further shown, phacoemulsification probe 12 includes a piezoelectric element such as a piezoelectric element 55 (e.g., one or more piezoelectric crystals) that drives needle 16 to vibrate, for example to vibrate in a resonant vibration mode that is used to break a cataract into small pieces during a phacoemulsification procedure. Console 28 comprises a piezoelectric drive module 30, coupled with the piezoelectric element, using electrical wiring running in cable 33.

Processor 38 further conveys processor-controlled driving signals via cable 33 to, for example, maintain needle 16 at a selected vibration amplitude. The drive module may be realized in hardware or software, for example, in a proportional-integral-derivative (PID) control architecture.

Processor 38 may receive user-based commands via a user interface 40. Examples of user-based commands may include: setting a vibration mode and/or an operating frequency of the piezoelectric element, setting or adjusting an irrigation and/or aspiration rate of the irrigation pump 24 and aspiration pump 26, respectively, setting or adjusting needle 16 stroke amplitude, turning on irrigation and/or aspiration, turning off irrigation and/or aspiration, and/or otherwise controlling the system.

In some examples, the physician uses afoot pedal (not shown) as a means of control. The foot pedal may be operable at a plurality of different positions, each associated with a different type of system activation. In an example, at an initial (non-stepped) position FP0, no modules are activated; at a first position FP1, irrigation is activated; at a second position FP2, irrigation and aspiration are activated; and at a third position FP3, irrigation, aspiration and vibration of the piezoelectric element are activated. Additionally, or alternatively to using a foot pedal, in some systems irrigation is provided (or adjusted, e.g., increased, reduced) in an automated manner, such as in response to activation of aspiration and/or in response to indications obtained by the system sensor(s).

Additionally, or alternatively to the user interface and/or to the foot pedal, processor 38 may receive user-based commands from controls located in a handle 21 of probe 12.

In an example, user interface 40 and display 36 may be integrated into a touch screen graphical user interface. Some or all of the functions of processor 38 may be combined in a single physical component or, alternatively, implemented using multiple physical components. These physical components may comprise hard-wired or programmable devices, or a combination of the two. In some examples, at least some of the functions of processor 38 may be carried out by suitable software stored in a memory 35. This software may be downloaded to a device in electronic form, over a network, for example. Alternatively, or additionally, the software may be stored in tangible, non-transitory computer-readable storage media, such as optical, magnetic, or electronic memory.

The system shown in FIG. 1 may include further elements, which are omitted for clarity of presentation. For example, physician 15 typically performs the procedure using a stereo microscope or magnifying glasses, neither of which are shown. Physician 15 may use other surgical tools in addition to probe 12, which are also not shown in order to maintain clarity and simplicity of presentation.

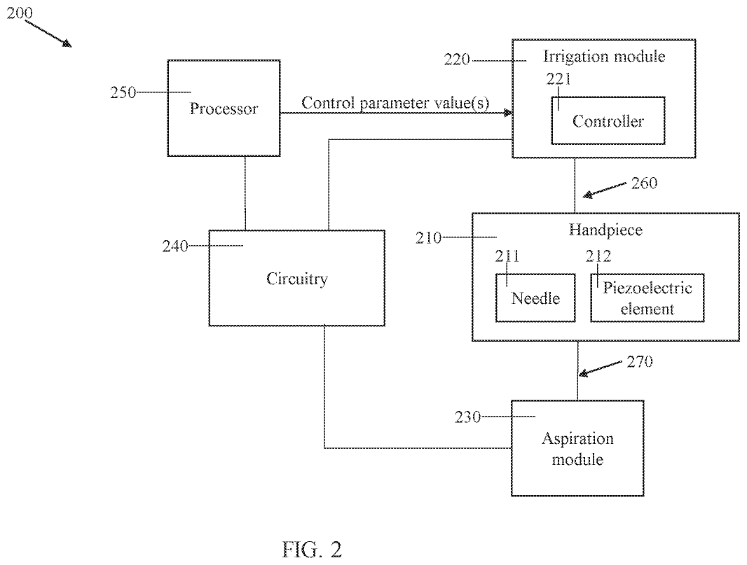

Reference is now made to FIG. 2, which is a simplified block diagram of a phacoemulsification system, according to examples of the present disclosure. FIG. 2 schematically illustrates how a feedback control loop may be established by using information about the flow of irrigation fluid and the aspiration flow to determine control parameter values for the irrigation module.

Phacoemulsification system 200 includes handpiece 210, irrigation module 220, aspiration module 230, circuitry 240 and processor 250.

Handpiece 210 includes needle 211 and piezoelectric element 212, as described above with reference to FIG. 1. Needle 211 is configured to be inserted into an eye and to be vibrated by piezoelectric element 212 to emulsify the lens of the eye.

Irrigation module 220 supplies the flow of irrigation fluid to the eye. The rate of flow of the irrigation fluid to the eye is based on the value(s) of at least one control parameter.

Aspiration module 230 aspirates material from the eye. The fluid removed by aspiration may comprise natural eye fluid, irrigation fluid, and lens material (e.g., the emulsified parts of the cataract).

Circuitry 240 determines the difference between the flow of irrigation fluid to the eye and aspiration flow of the material from the eye. For purposes of example, circuitry 240 is illustrated as being connected to irrigation module 220 and aspiration module 230 in order to obtain information regarding the irrigation fluid flow and the aspiration flow. In other examples, circuitry 240 may, alternately or additionally, be connected to other portions of phacoemulsification system 200 to obtain such information. For example, circuitry 240 may be connected to one or more flow sensors on irrigation channel 260 that conveys the irrigation fluid and/or on aspiration channel 270 that conveys the aspiration flow.

Based on the determined difference, processor 250 sets the value(s) of one or more control parameters and provides the control parameter values to irrigation module 220.

Optionally, circuitry 240 includes processing capabilities which calculate the difference in fluid flows (e.g., based on sensor readings), in which case a separate processing module (e.g., processor 250) may not be needed.

For clarity of presentation, irrigation channel 260 and aspiration channel 270 are illustrated schematically as connecting to handpiece 210, similarly to the non-limiting arrangement shown in FIG. 1.

Optionally, processor 250 sets the control parameter value(s) based on the average of the difference between the flow irrigation fluid and aspiration flow during a preceding time period. The duration of this time period may be relatively long in order to gradually adapt the irrigation fluid flow to the loss of fluid through the incision(s), for example one minute. Changing the irrigation fluid flow in a gradual manner prevents large and/or rapid changes in the irrigation flow rate which could risk the safety of the eye, but still allows for the system to compensate for fluid leakage to better manage the IOP. Further optionally, the duration of the time period is at least 60 seconds.

Optionally, processor 250 sets the respective values of the control parameter(s) based on information retrieved from a table. The table is referenced by the determined difference between irrigation fluid flow and aspiration flow. In one example, processor 250 looks up the control parameter value(s) directly from the table based on the index that is closest to the determined difference. In a second example, processor 250 uses an interpolation algorithm to estimate control parameter values from the closest indices above and below the determined difference. Alternately or additionally, processor 250 uses a formula to set some or all of the respective values of the control parameter(s).

Optionally, irrigation module 220 includes controller 221 which controls the flow of irrigation fluid based on the respective values of the control parameter(s). In optional examples, the controller is located on an internal real-time processor of phacoemulsification system 200. In alternate optional examples, the controller is part of an external device such as a computer (not shown).

Optionally, controller 221 controls a processor-controlled irrigation pump which pumps irrigation fluid into the eye from an irrigation reservoir or tank via an irrigation sleeve.

Optionally, the controller is a proportional-integral-derivative controller (PID) controller, and the parameter values provided by processor 250 are the values of the integral coefficient (Ki) and the derivative coefficient (Kd). Further optionally, processor 250 also provides the value of a proportional coefficient (Kp) to the PID controller.

Optionally, the flow of irrigation fluid and/or the aspiration flow is determined based on the rotation rate of the rotor of a respective positive displacement pump such as a progressing cavity pump (PCP) or a peristaltic pump. For example, if irrigation module 220 includes a PCP, the amount of irrigation fluid supplied to the eye over the preceding time period may be determined from the number of rotations of the rotor over that time period using an encoder.

Alternately or additionally, the flow of irrigation fluid and/or the aspiration flow is determined by a respective flow sensor.

Optionally, upon startup processor 250 initializes the irrigation fluid flow to compensate for the maximum expected leakage. Further optionally, the irrigation fluid flow is initialized for the worst case expected leakage of 13 cc/min. If the loss of fluid through the incision(s) is small, the flow of irrigation fluid will gradually reduce to a level that creates a lower, but still safe, IOP.

### II. Method for Controlling Irrigation in a Phacoemulsification System

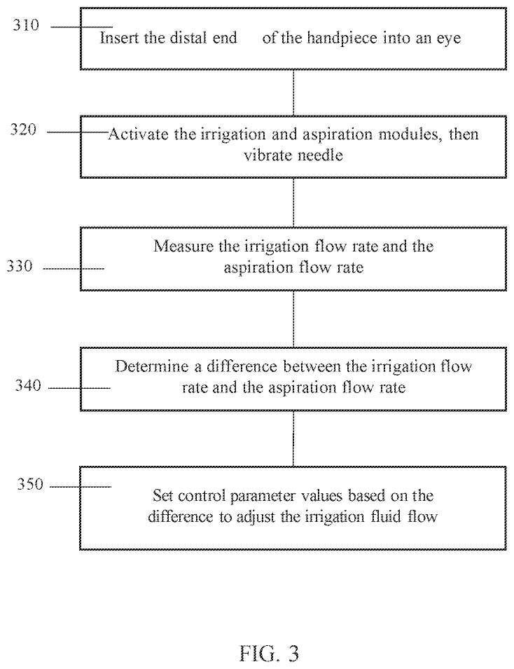

Reference is now made to FIG. 3, which is a flowchart of a method for controlling irrigation in a phacoemulsification system, according to examples of the present disclosure.

At 310, the distal end of the handpiece of a phacoemulsification system is inserted into an eye. The handpiece includes a piezoelectric element, a needle, a sleeve, an irrigation channel, and an aspiration channel.

At 320, The irrigation and aspiration modules are activated (for example by placing the foot pedal in the second position). After the irrigation and aspiration modules are activated, the needle is vibrated to emulsify the lens in the eye (for example by placing the foot pedal in the third position).

At 330, the irrigation flow rate and the aspiration flow rate are measured. Optionally, the irrigation flow rate and/or the aspiration flow rate are measured by an encoder, based on the rotation rate of the rotor of a respective positive displacement pump (e.g. a PCP or a peristaltic pump).

At 340, the difference between irrigation fluid flow through the irrigation channel and aspiration flow through the aspiration channel is determined.

At 350, the control parameter value(s) are set based on the difference determined at 340.

Optionally, the control parameter values are set based on the average level of the difference between irrigation fluid flow and aspiration flow during a preceding time period, as described above.

Optionally, the respective values of the control parameter(s) are set based on information retrieved from a table. Further optionally, an interpolation algorithm is used to estimate control parameter values from the closest indices above and below the determined difference.

Optionally, the control parameters are provided to a controller that controls the flow of irrigation fluid through the irrigation channel and the sleeve based on the control parameter values.

Optionally, the controller is a PID controller and the control parameter values set and provided to the PID controller are an integral coefficient (Ki) and a derivative coefficient (Kd). Further optionally, a parameter value for Kp is also set and provided to the PID controller.

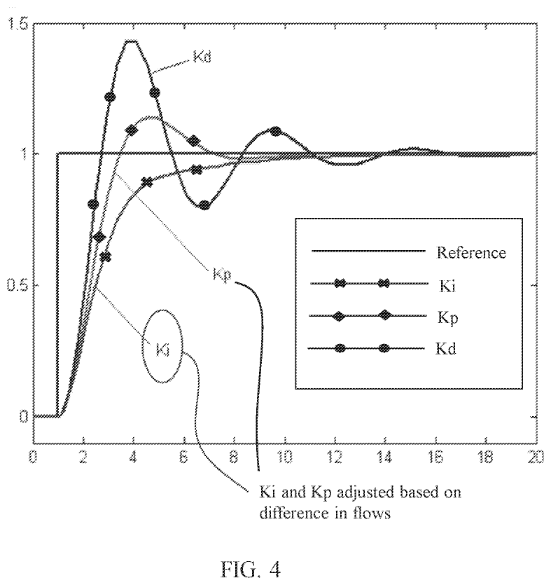

Reference is now made to FIG. 4, which is a simplified diagram illustrating the gradual adjustment of parameter values for a PID controller over time. The parameter values are based on the difference of irrigation fluid flow through the irrigation channel and the aspiration flow of the material through the aspiration channel. In cases where the leakage of fluid through the eye is relatively constant over time, the Ki, Kp and Kd parameters converge to steady state values.

### SUMMARY OF THE PRESENT DISCLOSURE

### Example 1

A phacoemulsification system (200), comprising:
a handpiece (210) comprising a piezoelectric element (55) and a needle (16), wherein the needle (16) is configured to be inserted into an eye and to be vibrated by the piezoelectric element (55) to emulsify a lens of the eye;
an irrigation module (220) configured to supply a flow of irrigation fluid to the eye based on respective values of at least one control parameter;
an aspiration module (230) configured for aspiration of material from the eye;
a circuitry (240) configured to determine a difference between the flow of irrigation fluid to the eye and an aspiration flow of the material from the eye; and
a processor (250) associated with the circuitry (240) and the irrigation module (220), configured to set the respective values of the at least one control parameter based on the determined difference, and to provide the control parameter values to the irrigation module (220).

### Example 2

The system (200) according to Example 1, wherein the processor (250) sets the respective values of the at least one control parameter based on an average of the determined difference during a preceding time period.

### Example 3

The system (200) according to Example 2, wherein a duration of the preceding time period is at least 60 seconds.

### Example 4

The system (200) according to any one of Examples 1-3, wherein the processor (250) is configured to set the respective values based on information retrieved from a table referenced by a determined difference.

### Example 5

The system (200) according to any one of Examples 1-4, wherein the irrigation module (220) comprises a controller (221) configured to control the flow of irrigation fluid based on the respective values of the at least one control parameter.

### Example 6

The system (200) according to Example 5, wherein the controller (221) is a proportional-integral-derivative controller (PID) controller, and the respective values comprises values of an integral coefficient (Ki) and a derivative coefficient (Kd).

### Example 7

The system (200) according to Example 6, wherein the processor (250) is further configured to provide a value of a proportional coefficient (Kp) to the PID controller.

### Example 8

The system (200) according to any one of Examples 1-7, wherein at least one of the flow of irrigation fluid and the aspiration flow is determined based on a rotation rate of a rotor of a respective positive displacement pump.

### Example 9

The system (200) according to any one of Examples 1-8, wherein at least one of the flow of irrigation fluid and the aspiration flow is determined by a respective flow sensor.

### Example 10

The system (200) according to any one of Examples 1-9, wherein upon startup the processor (250) initializes the irrigation fluid flow to compensate for a maximum expected leakage from the eye.

### Example 11

A method of operating a phacoemulsification system (200), comprising:
inserting into an eye at least a part of a handpiece (210) comprising a piezoelectric element (55), a needle (16), a sleeve, an irrigation channel, and an aspiration channel;
vibrating the needle (16) to emulsify a lens of the eye;
flowing irrigation fluid through the irrigation channel and the sleeve into the eye;
aspirating material from the eye through the aspiration channel;
determining, via system circuitry (240), a difference between irrigation fluid flow through the irrigation channel and aspiration flow of the material through the aspiration channel; and
via a system processor (250), setting respective values of at least one control parameter based on the determined difference and providing the at least one control parameter value to a controller (221), the controller (221) being configured to control the flow of irrigation fluid through the irrigation channel and the sleeve based on the at least one provided control parameter value.

### Example 12

The method according to Example 11, wherein the system processor (250) sets the respective values based on an average level of the determined difference during a preceding time period.

### Example 13

The method according to Example 11 or Example 12, wherein the controller (221) comprises a proportional-integral-derivative controller (PID) controller and the system processor (250) is configured to provide respective values of an integral coefficient (Ki) and a derivative coefficient (Kd) to the PID controller.

As such, those skilled in the art to which the present invention pertains, can appreciate that while the present invention has been described in terms of preferred examples, the concept upon which this disclosure is based may readily be utilized as a basis for the designing of other structures, systems and processes for carrying out the several purposes of the present invention.

The various illustrative logical blocks, modules, and algorithm steps described in connection with the examples disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing any departure from the scope of the disclosure.

It will also be understood that the system according to the present disclosure may be, at least partly, implemented on a suitably programmed computer. Likewise, the present disclosure contemplates a computer program being readable by a computer for executing the method of the invention. The present disclosure further contemplates a non-transitory computer-readable memory tangibly embodying a program of instructions executable by the computer for executing the method of the present disclosure.

Also, it is to be understood that the phraseology and terminology employed herein are for the purpose of description and should not be regarded as limiting.

It should be noted that the words “comprising”, “including” and “having” as used throughout the appended claims are to be interpreted to mean “including but not limited to”. The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases, and disjunctively present in other cases.

It is important, therefore, that the scope of the invention is not construed as being limited by the illustrative examples set forth herein. Other variations are possible within the scope of the present invention as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

1. A phacoemulsification system, comprising:
a handpiece comprising a piezoelectric element and a needle, wherein the needle is configured to be inserted into an eye and to be vibrated by the piezoelectric element to emulsify a lens of the eye;
an irrigation module configured to supply a flow of irrigation fluid to the eye based on respective values of at least one control parameter;
an aspiration module configured for aspiration of material from the eye;
a circuitry configured to determine a difference between the flow of irrigation fluid to the eye and an aspiration flow of said material from the eye; and
a processor associated with said circuitry and said irrigation module, configured to set said respective values of said at least one control parameter based on said determined difference, and to provide said control parameter values to said irrigation module.

2. The system according to claim 1, wherein said processor sets said respective values of said at least one control parameter based on an average of said determined difference during a preceding time period.

3. The system according to claim 2, wherein a duration of said preceding time period is at least 60 seconds.

4. The system according to claim 1, wherein said processor is configured to set said respective values based on information retrieved from a table referenced by a determined difference.

5. The system according to claim 1, wherein the irrigation module comprises a controller configured to control said flow of irrigation fluid based on said respective values of said at least one control parameter.

6. The system according to claim 5, wherein the controller is a proportional-integral-derivative controller (PID) controller, and said respective values comprises values of an integral coefficient (Ki) and a derivative coefficient (Kd).

7. The system according to claim 6, wherein said processor is further configured to provide a value of a proportional coefficient (Kp) to said PID controller.

8. The system according to claim 1, wherein at least one of said flow of irrigation fluid and said aspiration flow is determined based on a rotation rate of a rotor of a respective positive displacement pump.

9. The system according to claim 1, wherein at least one of said flow of irrigation fluid and said aspiration flow is determined by a respective flow sensor.

10. The system according to claim 1, wherein upon startup said processor initializes said irrigation fluid flow to compensate for a maximum expected leakage from said eye.

11. A method of operating a phacoemulsification system, comprising:
inserting into an eye at least a part of a handpiece comprising a piezoelectric element, a needle, a sleeve, an irrigation channel, and an aspiration channel;
vibrating the needle to emulsify a lens of the eye;
flowing irrigation fluid through the irrigation channel and the sleeve into the eye;
aspirating material from the eye through the aspiration channel;
determining, via system circuitry, a difference between irrigation fluid flow through said irrigation channel and aspiration flow of said material through said aspiration channel; and
via a system processor, setting respective values of at least one control parameter based on said determined difference and providing said at least one control parameter value to a controller, said controller being configured to control said flow of irrigation fluid through the irrigation channel and the sleeve based on said at least one provided control parameter value.

12. The method of claim 11, wherein said system processor sets said respective values based on an average level of said determined difference during a preceding time period.

13. The method of claim 11, wherein said controller comprises a proportional-integral-derivative controller (PID) controller and said system processor is configured to provide respective values of an integral coefficient (Ki) and a derivative coefficient (Kd) to said PID controller.
